(19) United States
(12) Patent Application Publication
(10) Pub. No.: US 2014/0121519 A1
(43) Pub. Date: May 1, 2014

(54) ULTRASONIC DIAGNOSTIC APPARATUS AND ULTRASONIC DIAGNOSTIC APPARATUS CONTROL METHOD

(71) Applicants: Toshiba Medical Systems Corporation, Otawara-shi (JP); KABUSHIKI KAISHA TOSHIBA, Minato-ku (JP)

(72) Inventors: Yasuo MIYAJIMA, Utsunomiya-shi (JP); Nobuyuki Iwama, Nasushiobara-shi (JP); Isao Uchiumi, Nasushiobara-shi (JP); Masaaki Ishitsuka, Nasushiobara-shi (JP); Toru Hirano, Otawara-shi (JP); Hironobu Hongou, Otawara-shi (JP)

(73) Assignees: Toshiba Medical Systems Corporation, Otawara-shi (JP); KABUSHIKI KAISHA TOSHIBA, Minato-ku (JP)

(21) Appl. No.: 14/147,343

(22) Filed: Jan. 3, 2014

Related U.S. Application Data
(63) Continuation of application No. PCT/JP2012/067238, filed on Jul. 5, 2012.

(30) Foreign Application Priority Data
Jul. 5, 2011 (JP) 2011-149343
Jul. 4, 2012 (JP) 2012-150297

(51) Int. Cl. A61B 8/08; A61B 8/00; A61B 8/06; A61B 8/14

(52) U.S. Cl. CPC A61B 8/5207; A61B 8/145; A61B 8/4444; A61B 8/4483; A61B 8/463; A61B 8/54; A61B 8/06; A61B 8/469
USPC 600/440; 600/447

(57) ABSTRACT

According to one embodiment, an ultrasonic diagnostic apparatus comprises an ultrasonic probe, a transmission unit, a reception unit, a correction unit, an image processing unit and a display unit. The ultrasonic probe includes a plurality of ultrasonic transducer elements. The reception unit stores a plurality of image generation reception signals corresponding to the plurality of ultrasonic transducer elements, generates a reception beam in accordance a predetermined reception condition by using each of the stored image generation reception signals, and stores a plurality of correction reception signals corresponding to the plurality of ultrasonic transducer elements. The correction unit receives the plurality of correction reception signals and corrects at least one of the transmission conditions and the reception condition based on the plurality of correction reception signals.

### CROSS-REFERENCE TO RELATED APPLICATION

This application is based upon and claims the benefit of priority from Japanese Patent Applications No. 2011-149343, filed Jul. 5, 2011, and No. 2012-150297, filed Jul. 4, 2012, the entire contents of all of which are incorporated herein by reference.

### FIELD

Embodiments described herein relate generally to a medical ultrasonic diagnostic apparatus equipped with a digital beam former and an ultrasonic diagnostic apparatus control method implemented in the ultrasonic diagnostic apparatus.

### BACKGROUND

An ultrasonic diagnostic apparatus is a diagnostic apparatus which displays an image of in vivo information. This apparatus is used as a useful apparatus for noninvasive real-time observation at low cost without exposure to radiation as compared with other types of image diagnostic apparatuses such as an X-ray diagnostic apparatus and an X-ray computed tomography apparatus. Ultrasonic diagnostic apparatuses are used in a wide application range including diagnosis of circulatory organs such as the heart, abdominal regions such as the liver and kidney and peripheral vessels, diagnosis in obstetrics and gynecology, and breast cancer diagnosis.

Such an ultrasonic diagnostic apparatus acquires ultrasonic data from a tissue in a scanned region (a two-dimensional region or three-dimensional region) in an object by forming the directivity of a transmission/reception beam using an array transducer and sequentially changing the direction of the beam. The apparatus generates and displays a plurality of tomographic images, volume rendering images, or the like corresponding to the scanned region based on the obtained ultrasonic data. An observer comprehends a tissue shape in the object by observing the displayed images, and uses the images for diagnosis.

The resolution of an ultrasonic image depends on the width of an ultrasonic band in the distance direction, and depends on the frequency, the distance, the width of a transmission/reception aperture in the azimuth direction. According to this property, when, for example, an image is to be formed through a homogenous medium, the resolution increases as the aperture increases. In about 1980, an ultrasonic probe with an aperture of 30 to 40 elements was the mainstream. Nowadays, however, an ultrasonic probe with an increased aperture of about 100 elements has becomes the mainstream. Further increasing the aperture of an ultrasonic probe allows to expect to obtain images through homogenous media. On the other hand, in a practical application to obtain images through the skin, even increasing the aperture will not increase the resolution because of the inhomogeneity of acoustic properties of a subcutaneous tissue or the like. This rather leads to a situation in which unnecessary responses increase to output images unsuitable for diagnosis.

Recently, studies have been made on a technique of measuring biological inhomogeneity and correcting phase and amplitude distortions due to the inhomogeneity.

FIG. 12 is a block diagram showing the flows of reception signals in a conventional reception circuit. As shown in FIG. 12, signals from the respective ultrasonic transducer elements are digitized by A/D conversion after preprocessing and are sent to a beam former. In this case, memories 801 to 804 give the respective signals with delays on a clock basis, and digital filters 811 to 814 give the resultant signals with fine delays equal to or less than clocks, thereby adjusting the signals from a predetermined direction and depth so as to make them arrive at the same time. An addition circuit 821 adds the signals to form directivity. When performing simultaneous reception upon providing a plurality of predetermined directions, the circuit is configured to perform this delay/addition processing a plurality of number of times. On the other hand, in a processing system 9 which optimizes transmission/reception conditions for the digitized signals independently of the beam former, a circuit 921 analyzes the correlations between adjacent elements, estimates the inhomogeneity of a medium reaching the respective elements, and calculates correction values for amplitudes and delays of transmission/reception by using memories 901 to 903 which store the waveforms of the respective elements and correlation circuits 911 to 912 which calculate the correlations between outputs from the memories.

FIG. 13 is a view for explaining the operation of the memory of a conventional reception circuit. As shown in FIG. 13, a signal from each ultrasonic transducer element is digitized by A/C conversion after preprocessing and sent to the beam former (RX01, RX02).

The reception signal RX01 is sequentially written in a corresponding address region 801B of the memory 801, and the reception signal RX02 corresponding to the next transmission is written in a different address region 801C. Setting a wide address space so as to avoid overwriting by the next signal in this manner allows to read one reception signal a plurality of number of times. For example, it is possible to form different focuses and directivities by reading out the data of RX01 as RX01B1, RX01B2, and RX01B3 with different delay times. Such a function can improve the real-time performance when implementing three-dimensional scanning using two-dimensional array transducers. Read and write clock speeds for the memory are determined as follows. A write speed is determined by the band of acoustic signals. Thinning out information in advance at a write speed of about 40 to 60 MHz to set a state in which write operation is performed with lower-speed clocks. The read speed can be set to 100 MHz or more. In many cases, this allows to perform read operation a plurality of number of times and spend much time for read operation.

The conventional ultrasonic diagnostic apparatus, however, needs to be provided with a processing system for optimizing transmission/reception conditions separately from the beam former. This increases the circuit size and leads to increases in the size and cost of the apparatus, thereby avoiding the provision of a practical apparatus. These problems especially noticeable in a two-dimensional array ultrasonic probe.

In consideration of the above situation, it is an object to provide an ultrasonic diagnostic apparatus and ultrasonic diagnostic apparatus control method which can optimize transmission/reception conditions by receiving and analyzing signals from elements simultaneously with the acquisition of an image.

### SOLUTION TO PROBLEM

In order to achieve the above object, the following measures are taken.

An ultrasonic diagnostic apparatus according to an embodiment comprises an ultrasonic probe including a plurality of ultrasonic transducer elements configured to transmit ultrasonic waves to an object in response to supplied driving signals and generate reception signals based on reflected waves from the object; a transmission unit configured to supply the driving signals to the plurality of ultrasonic transducer elements in accordance with a predetermined transmission condition; a reception unit configured to store a plurality of image generation reception signals corresponding to the plurality of ultrasonic transducer elements, generate a reception beam in accordance a predetermined reception condition by using each of the stored image generation reception signals, and store a plurality of correction reception signals corresponding to the plurality of ultrasonic transducer elements; a correction unit configured to receive the plurality of correction reception signals and correct at least one of the transmission condition and the reception condition based on the plurality of correction reception signals; an image processing unit configured to generate an ultrasonic image based on the reception beam; and a display unit configured to display the ultrasonic image.

### BRIEF DESCRIPTION OF DRAWINGS

FIG. 1 is a block diagram of an ultrasonic diagnostic apparatus 1 according to an embodiment.

FIG. 2 is a block diagram showing the arrangement of an ultrasonic reception unit 22.

FIG. 3 is a conceptual view for explaining a processing procedure in a beam forming processing system and an analysis correction processing system in a predetermined channel.

FIG. 4 is a view for explaining a transmission/reception condition optimization function.

FIG. 5 is a view for explaining the transmission/reception condition optimization function.

FIG. 6A is a view for explaining an application example 1 of the transmission/reception condition optimization function.

FIG. 6B is a view for explaining the application example 1 of the transmission/reception condition optimization function.

FIG. 6C is a view for explaining the application example 1 of the transmission/reception condition optimization function.

FIG. 7A is a view for explaining an application example 2 of the transmission/reception condition optimization function.

FIG. 7B is a view for explaining the application example 2 of the transmission/reception condition optimization function.

FIG. 7C is a view for explaining the application example 2 of the transmission/reception condition optimization function.

FIG. 8A is a view for explaining an application example 3 of the transmission/reception condition optimization function.

FIG. 8B is a view for explaining the application example 3 of the transmission/reception condition optimization function.

FIG. 9 is a view for explaining an application example 4 of the transmission/reception condition optimization function.

FIG. 10A is a view for explaining the application example 4 of the transmission/reception condition optimization function.

FIG. 10B is a view for explaining the application example 4 of the transmission/reception condition optimization function.

FIG. 10C is a view for explaining the application example 4 of the transmission/reception condition optimization function.

FIG. 11 is a view for explaining the second embodiment using a two-dimensional array probe.

FIG. 12 is a view for explaining reception processing in a conventional ultrasonic diagnostic apparatus.

FIG. 13 is a view for explaining the operation of a memory of a conventional reception circuit.

### DESCRIPTION OF EMBODIMENTS

In general, according to one embodiment, an ultrasonic diagnostic apparatus comprises an ultrasonic probe, a transmission unit, a reception unit, a correction unit, an image processing unit and a display unit. The ultrasonic probe includes a plurality of ultrasonic transducer elements configured to transmit ultrasonic waves to an object in response to supplied driving signals and generate reception signals based on reflected waves from the object. The transmission unit supplies the driving signals to the plurality of ultrasonic transducer elements in accordance with a predetermined transmission condition. The reception unit stores a plurality of image generation reception signals corresponding to the plurality of ultrasonic transducer elements, generates a reception beam in accordance a predetermined reception condition by using each of the stored image generation reception signals, and stores a plurality of correction reception signals corresponding to the plurality of ultrasonic transducer elements. The correction unit receives the plurality of correction reception signals and corrects at least one of the transmission conditions and the reception condition based on the plurality of correction reception signals. The image processing unit generates an ultrasonic image based on the reception beam. The display unit displays the ultrasonic image.

The first and second embodiments will be described below with reference to the accompanying drawing. Note that the same reference numerals in the following description denote constituent elements having almost the same functions and arrangements, and a repetitive description will be made only when required.

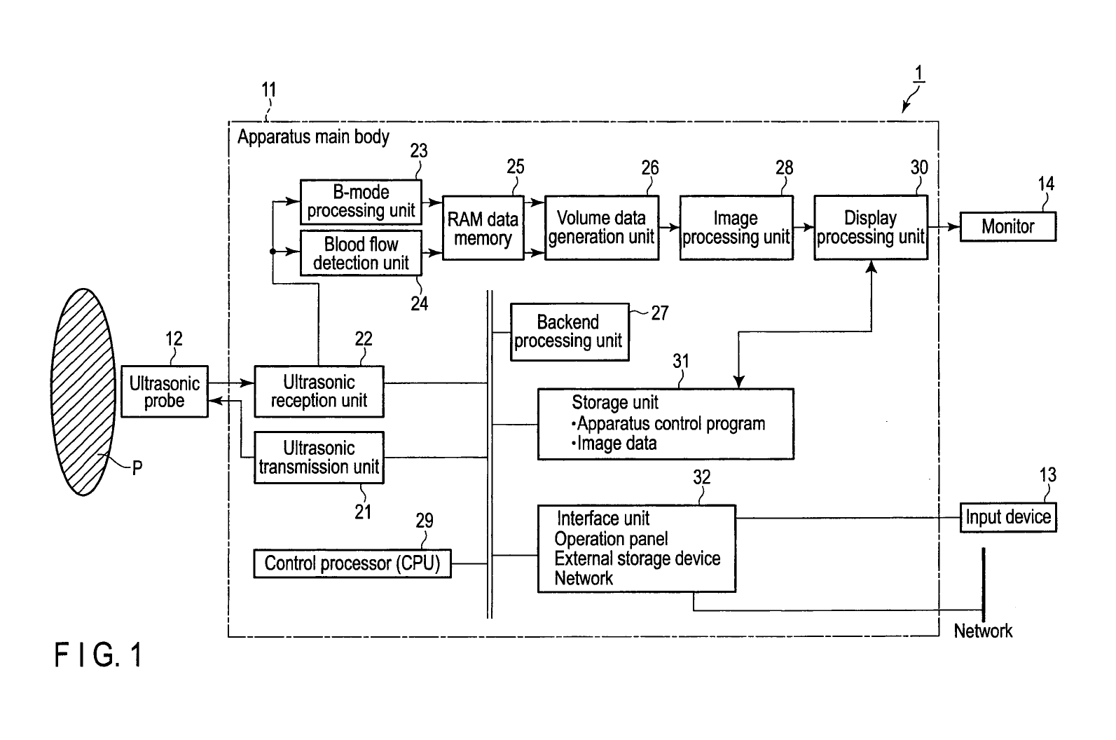

FIG. 1 is a block diagram of an ultrasonic diagnostic apparatus 1 according to this embodiment. As shown in FIG. 1, the ultrasonic diagnostic apparatus 1 includes an ultrasonic probe 12, an input device 13, a monitor 14, an ultrasonic transmission unit 21, an ultrasonic reception unit 22, a B-mode processing unit 23, a Doppler processing unit 24, a RAW data memory 25, a volume data generation unit 26, an image processing unit 28, a display processing unit 30, a control processor 29, a storage unit 31, and an interface unit 32.

The ultrasonic probe 12 is a device (probe) which transmits ultrasonic waves to an object and receives reflected waves from the object based on the transmitted ultrasonic waves. The ultrasonic probe 12 has, on its distal end, an array of a plurality of piezoelectric transducers, a matching layer, a backing member, and the like. The ultrasonic probe 12 is connected to an ultrasonic diagnostic apparatus main body 11 via a cable. Each ultrasonic transducer forms an independent channel, transmits an ultrasonic wave in a desired direction in a scan area based on a driving signal from the ultrasonic transmission unit 21, and converts a reflected wave from the object into an electrical signal. The matching layer is an intermediate layer which is provided for the piezoelectric transducers to make ultrasonic energy efficiently propagate. The backing member prevents ultrasonic waves from propagating backward from the piezoelectric transducers. When the ultrasonic probe 12 transmits an ultrasonic wave to an object P, the transmitted ultrasonic wave is sequentially reflected by a discontinuity surface of acoustic impedance of internal body tissue, and is received as an echo signal by the ultrasonic probe 12. The amplitude of this echo signal depends on an acoustic impedance difference on the discontinuity surface by which the echo signal is reflected. The echo produced when a transmitted ultrasonic pulse is reflected by a moving blood flow is subjected to a frequency shift depending on the velocity component of the moving body in the ultrasonic transmission/reception direction due to the Doppler effect.

Note that the ultrasonic probe 12 according to this embodiment may be either a one-dimensional probe (i.e., a probe having a plurality of ultrasonic transducers arranged along one direction) or a two-dimensional array probe (i.e., a probe having ultrasonic transducers arranged in the form of a two-dimensional matrix).

The input device 13 is connected to an apparatus main body 11 and includes various types of switches, buttons, a trackball, a mouse, and a keyboard which are used to input, to the apparatus main body 11, various types of instructions, conditions, an instruction to set a region of interest (ROI), various types of image quality condition setting instructions, and the like from an operator.

The monitor 14 displays morphological information and blood flow information in the living body as images based on video signals from a display processing unit 27.

The ultrasonic transmission unit 21 includes a clock generator, a frequency divider, a transmission delay circuit, and a pulser. The frequency divider decreases the clock pulse generated by the clock generator to a rate pulse of about 5 kHz. This rate pulse is supplied to the pulser via the transmission delay circuit to generate a high-frequency voltage pulse, thereby driving each ultrasonic transducer of the ultrasonic probe 12 (i.e., mechanically vibrates each ultrasonic transducer). The ultrasonic transmission unit 21 may be configured to generate an arbitrary waveform. The ultrasonic wave transmitted into the object via the ultrasonic probe 12 is reflected by an acoustic impedance boundary in the living body. Each ultrasonic transducer then converts mechanical vibration into an electrical signal based on the reflected wave.

The ultrasonic reception unit 22 receives the electrical signal originating from the reflected wave from each ultrasonic transducer and performs predetermined processing for the signal, thereby generating a signal having directivity (echo signal). The ultrasonic reception unit 22 also analyzes the inhomogeneity of the object (living body) and executes processing for correcting it. The arrangement of the ultrasonic reception unit 22 will be described in detail later.

The B-mode processing unit 23 receives an echo signal from the reception unit 22, and performs logarithmic amplification, envelope detection processing, and the like for the signal to generate data whose signal intensity is expressed by a luminance level.

The Doppler processing unit 24 is a unit which implements so-called color Doppler imaging (CDI). First of all, the Doppler processing unit 24 extracts a Doppler signal having undergone a frequency shift from the echo signal from a reception circuit 5. An MTI filter then transmits only a specific frequency component of the extracted Doppler signal. An autocorrelator obtains the frequency of the signal having passed through the filter. A computation unit computes a mean velocity, variance, and power from this frequency. Note that adjusting the pass band of the MTI filter makes it possible to switch between a general Doppler mode (image data obtained in this mode will be referred to as blood flow Doppler image data) of mainly visualizing a blood flow and a tissue Doppler mode (image data obtained in this mode will be referred to as tissue Doppler image data) of mainly visualizing an organ such as the cardiac muscle.

The raw data memory 25 generates B-mode raw data as B-mode data on ultrasonic scanning lines by using a plurality of B-mode data received from the B-mode processing unit 23. The raw data memory 25 generates blood flow raw data as blood flow data on three-dimensional ultrasonic scanning lines by using a plurality of blood flow data received from the blood flow detection unit 24. Note that for the purpose of reducing noise or smooth concatenation of images, a three-dimensional filter may be inserted after the raw data memory 25 to perform spatial smoothing.

The volume data generation unit 26 generates B-mode volume data from the B-mode raw data received from the RAW data memory 25 or the volume data generation unit 26 by converting raw data into a volume-based data arrangement. This conversion is performed to generate B-mode volume data on each visual line in a view volume used in image generation processing by processing in consideration of spatial position information. Note that this embodiment has exemplified the case in which various types of processing are executed by using the B-mode volume data generated by the above conversion processing. However, the embodiment is not limited to this case, and may execute processing based on a high-resolution data acquisition function using the B-mode voxel volume generated by executing raw-voxel conversion.

A backend processing unit 27 analyzes arrival time differences between elements, phase distortions, amplitude distortions, and the like by using reception signals from the respective ultrasonic transducer elements under the control of the control processor 29, and corrects transmission delays and reception delays based on these results.

The image processing unit 28 performs predetermined image processing such as volume rendering, MPR (Multi Planar Reconstruction), and MIP (Maximum Intensity Projection) by using the data received from the volume data generation unit 26. Note that for the purpose of reducing noise or smooth concatenation of images, a two-dimensional filter may be inserted after the image processing unit 28 to perform spatial smoothing.

The display processing unit 30 executes various kinds of processes associated with a dynamic range, luminance (brightness), contrast, y curve correction, RGB conversion, and the like for various kinds of image data generated/processed by the image processing unit 28.

The control processor 29 has the function of an information processing apparatus (computer) and controls the operation of the main body of this ultrasonic diagnostic apparatus. In particular, the control processor 29 controls the ultrasonic reception unit 22 to time-divisionally store an image generation echo signal and a plurality of correction echo signals in the transmission/reception condition optimization function. Alternatively, the control processor 29 controls the reception unit 22 to store an image generation echo signal and a plurality of correction echo signals in physically different memory areas and output the respective signals at different timings. With these control operations, the reception unit 22 implements the following two functions at predetermined timings under the control of the control processor 29: the first function of outputting an echo signal corresponding to an image generation reception beam; and the second function of outputting a correction echo signal.

The storage unit 31 stores diagnosis information (patient ID, findings by doctors, and the like), a diagnostic protocol, transmission/reception conditions, an image processing program, a body mark generation program, a dedicated program for implementing the transmission/reception condition optimization function (to be described later), and other data groups. The storage unit 31 is also used to store images in the image memory (not shown), as needed. It is possible to transfer data in the storage unit 31 to an external peripheral device via the interface unit 32.

The interface unit 32 is an interface associated with the input device 13, a network, and a new external storage device (not shown). The interface unit 32 can transfer, via a network, data such as ultrasonic images, analysis results, and the like obtained by this apparatus to another apparatus.

### (Transmission/Reception Condition Optimization Function)

The transmission/reception condition optimization function of the ultrasonic diagnostic apparatus 1 will be described next. This function analyzes the inhomogeneity of an ultrasonic propagation medium by receiving and using an echo signal while acquiring an echo single for each channel, and optimizes transmission/reception conditions without increasing the circuit size. The ultrasonic reception unit 22 executes the transmission/reception condition optimization function under the control of the control processor 29.

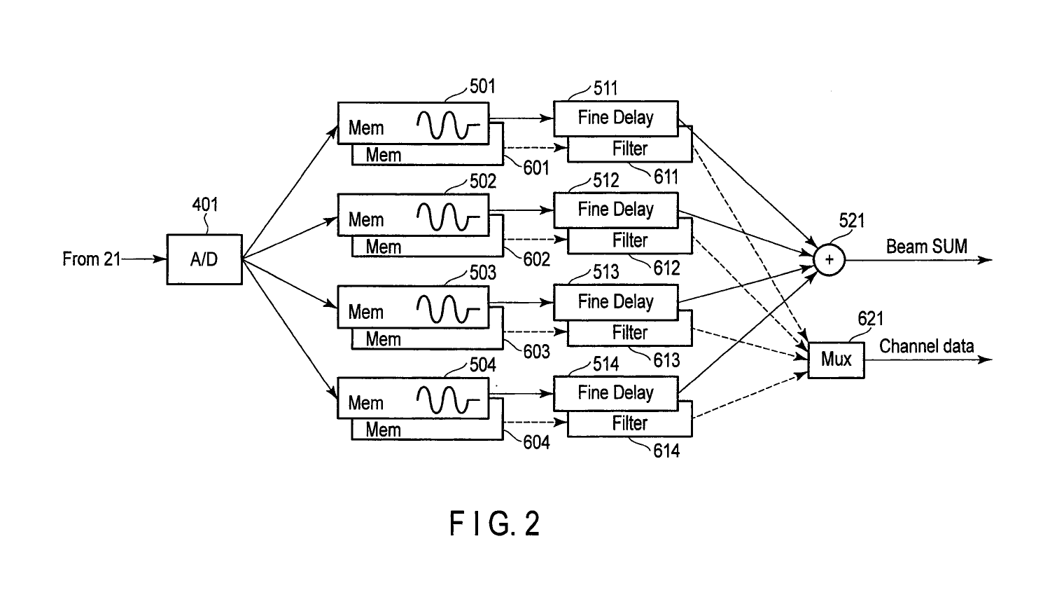

FIG. 2 is a block diagram of the ultrasonic reception unit 22. As shown in FIG. 2, the ultrasonic reception unit 22 includes an A/D converter 401, beam former memories 501 to 504, FIR filters 511 to 514, an adder 521, independent memories 601 to 604, filters 611 to 614, and a multiplexer 621. The beam former memories 501 to 504, the FIR filters 511 to 514, and the adder 521 constitute a beam forming processing system. The independent memories 601 to 604, the filters 611 to 614, and the multiplexer 621 constitute a processing system (analysis/correction processing system) for implementing the transmission/reception condition optimization function. For the sake of easy explanation, FIG. 2 exemplifies an arrangement corresponding to four channels (four ultrasonic transducer elements). In practice, however, signal processing systems are individually provided for the respective ultrasonic transducer elements.

As shown in FIG. 2, the A/D converter 401 digitizes echo signals from the respective ultrasonic transducer elements after preprocessing, and sends the resultant signals to the beam former memories 501 to 504. The beam former memories 501 to 504 execute delays (relatively long delays) on a clock basis. That is, in write or read operation with respect to the beam former memories 501 to 504, differently performing address control for the respective elements will give delays to signals for each unit of sampling when digitizing them. Thereafter, the FIR filters 511 to 514 execute further correct delay processing by giving the signals with fine delays equal to or less than a clock. Echo signals corresponding to the respective channels are adjusted by these delay processes so as to make them from a predetermined direction/depth arrive at the same time. The addition circuit 521 adds the respective echo signals after the respective delay processes, thereby forming a reception beam having high directivity. Note that the addition circuit 521 has an arrangement for forming different directivities for simultaneously and selectively extracting signals from a plurality of directions and an arrangement for repetitive reading.

In addition, the A/D converter 401 digitizes echo signals from the respective ultrasonic transducer elements after preprocessing, and simultaneously stores the resultant signals in the independent memories 601 to 604 concurrently with input processing to the beam former. The independent memories 601 to 604 constitute a system logically independent of beam forming. It is however possible to physically isolate the areas of the beam former memories 501 to 504 and store signals in the physical memories. The filters 611 to 614 filter the respective echo signals read out from the independent memories 601 to 604 and extract signals in a predetermined band. The multiplexer 621 generates channel data for correcting amplitudes/delays for the respective channels and outputs the data to the processing system on the subsequent stage (the backend processing unit 27 and the like). The control processor 29 estimates the inhomogeneity of paths through which ultrasonic waves reach the respective ultrasonic transducers in the object by analyzing the cross-correlations between channel data (echo signals in a predetermined band) for the respective channels from the multiplexer 621 (the correlations between signals from adjacent ultrasonic transducers).

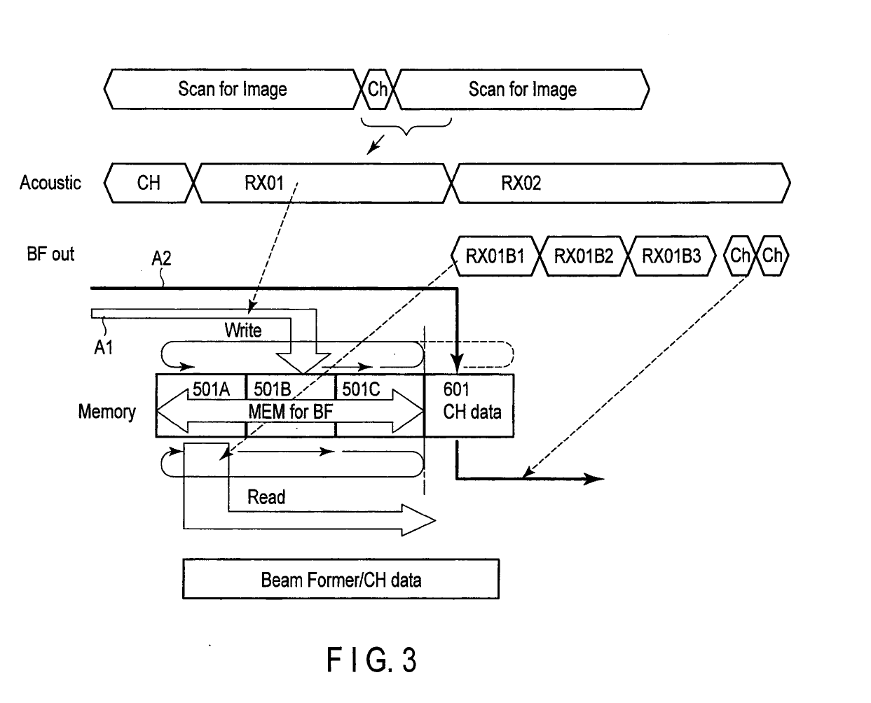

FIG. 3 is a conceptual view for explaining a processing procedure in the beam forming processing system and the analysis/correction processing system in a predetermined channel (i.e., a predetermined ultrasonic transducer element).

As indicated by an arrow A1 in FIG. 3, when the beam former memory 501 receives digitized echo signals RX01 and RX02 after preprocessing performed in accordance with a predetermined channel, the reception signal RX01 corresponding to (temporally preceding) previous transmission TX01 is sequentially written in an address region 501B corresponding to the beam former memory 501. The reception signal RX02 corresponding to next transmission TX02 is written in an address region 501C different from the address region 501B. That is, before delays, the memory 501 stores simultaneously acquired signals upon dividing them into packets gated in the distance direction.

On the other hand, the data of the ultrasonic transducer elements used for inhomogeneity analysis/correction processing are written in the independent memory 601 and held independently of beam forming CH data, as indicated by an arrow A2 in FIG. 3.

In beam forming, one reception signal (e.g., the data of RX01) is read three times in RX01B1, RX01B2, and RX01B3 with different delay times. In addition, CH data corresponding to the ultrasonic transducer elements are sent to the backend processing unit 27 via a filter and the like along the same path as that for beam former outputs in the remaining time. Thereafter the control processor 29 executes correlation calculation associated with the ultrasonic transducer elements by using a signal for each channel stored in the backend processing unit 27, thereby executing inhomogeneity analysis on a propagation medium while displaying an image on the subsequent processing system.

If transmission/reception ultrasonic waves have gone through a homogeneous propagation medium, signals from the respective ultrasonic transducer elements arrive with delay times corresponding to propagation distance differences as indicated by, for example, the left side of FIG. 4. If, therefore, signals from the respective ultrasonic transducer elements are delayed by predetermined delay times calculated from the propagation distances, the phases of the signals can be matched with each other, as indicated by the right side of FIG. 4. Adding these signals can enhance a signal from a predetermined direction/distance and suppress signals from other places.

If transmission/reception ultrasonic waves have gone through an inhomogeneous propagation medium, signals from the respective ultrasonic transducer elements arrive at times difference from delay times corresponding to propagation distance differences as indicated by, for example, the left side of FIG. 5. For this reason, even delaying the signals from the respective ultrasonic transducer elements by predetermined delay times calculated from the propagation distances cannot match the phases of the signals, as indicated by the right side of FIG. 5. Even if, therefore, these signals are added, the enhancement of a signal from a predetermined direction/distance decreases, and the suppression of signals from other places reduces, leading to a deterioration in directivity and a decrease in the resolution of the image.

According to this transmission/reception condition optimization function, the backend processing unit 27 analyzes the arrival time differences between signals at the respective ultrasonic transducer elements and also analyzes phase distortions and amplitude distortions, thereby correcting transmission delays and reception delays based on these results. This makes it possible to properly reduce a deterioration in directivity.

### APPLICATION EXAMPLE 1

Another application example of this transmission/reception condition optimization function will be described next. This application example is configured to analyze an amplitude for each ultrasonic transducer element and properly perform sensitivity adjustment. Note that this apparatus acquires the waveforms of signals from the respective ultrasonic transducer elements while performing beam forming for displaying an image, and transfers the resultant signals to the backend processing unit 27 in the same manner as described above.

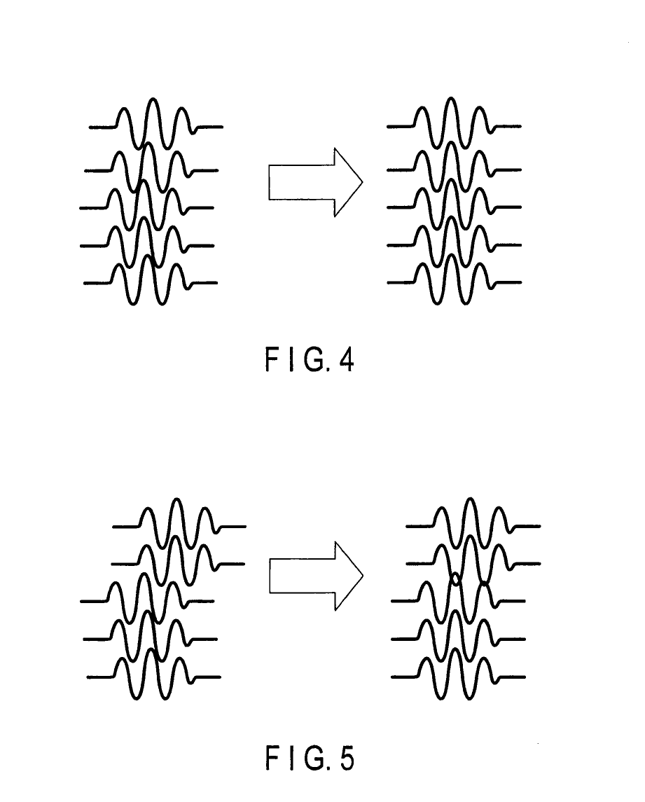

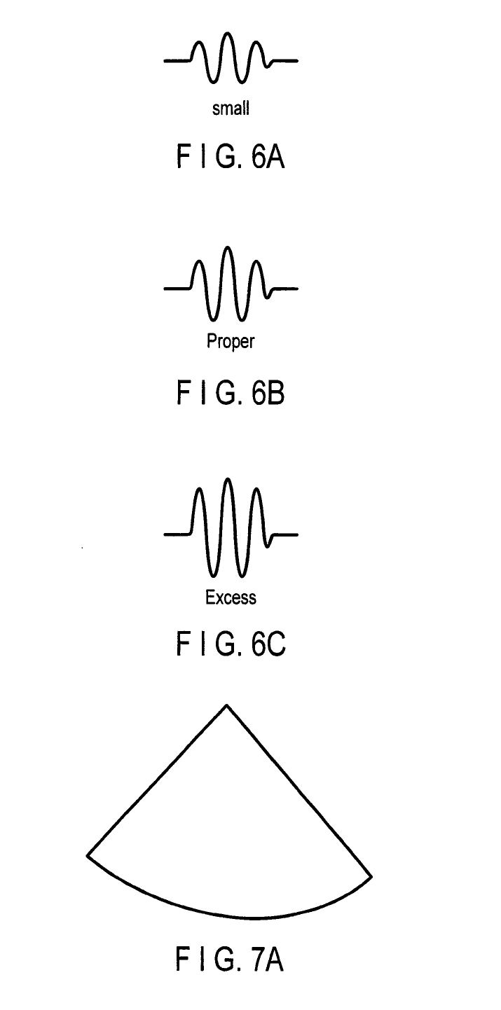

The transmission/reception condition optimization function according to this application example corrects reception sensitivity by analyzing the amplitudes of the signals acquired from the respective ultrasonic transducer elements in the following manner. That is, the backend processing unit 27 checks whether each signal is too small in amplitude as shown in FIG. 6A or too large in amplitude and has a saturated waveform as shown in FIG. 6C. If a signal is too small, the backend processing unit 27 increases the sensitivity, and the vice versa, thereby correcting the waveform to a proper waveform like that shown in FIG. 6B.

### APPLICATION EXAMPLE 2

The following is an example of detecting the movement of the ultrasonic probe 12 relative to an imaging target by using this transmission/reception condition optimization function.

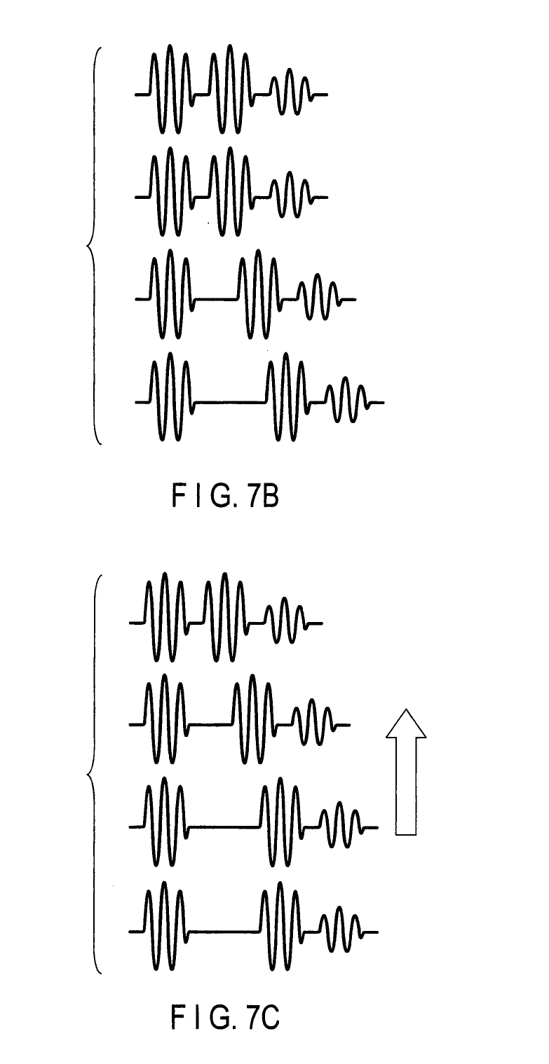

FIG. 7A shows an image example of a sector scanning type. The image acquired by this scanning scheme exhibits a narrow field of view at short distances. This makes it difficult to detect a change in short-distance image due to the movement of the ultrasonic probe 12. As shown in FIG. 7B, the apparatus detects a temporal change in the signal received from each ultrasonic transducer element and detects the movement of the ultrasonic probe 12 based on the detection result. When, for example, the ultrasonic probe 12 is moved in the direction of an arrow in FIG. 7C, the detected signals exhibit patterns representing the movement like that shown in FIGS. 7B and 7C. The backend processing unit 27 detects the movement of the ultrasonic probe 12 based on temporal changes in the signal patterns. Assume that the apparatus determines based on a movement detection result that there is no movement of the ultrasonic probe 12. In this case, it is also possible to provide an image with a high real-time property by making all the processing systems of the ultrasonic reception unit 22 operate as beam formers.

### APPLICATION EXAMPLE 3

The following is an application example of detecting the floating of the ultrasonic probe 12 from an object surface based on the amount of multiple reflection immediately under the ultrasonic transmission/reception surface of the ultrasonic probe 12.

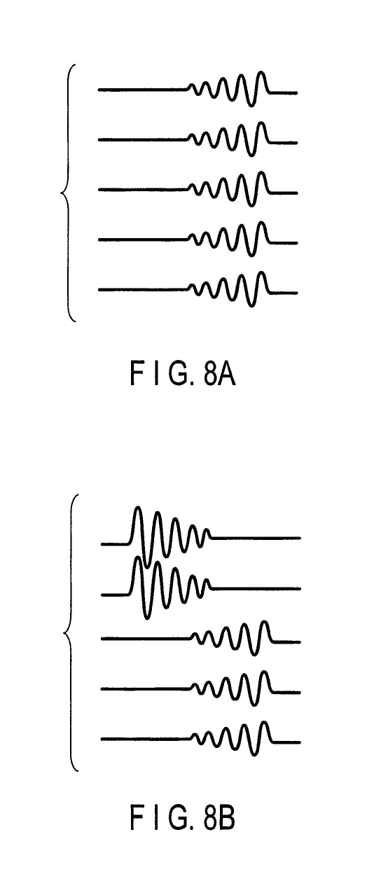

As shown in FIG. 8A, while the ultrasonic probe 12 does not float from the object surface, the magnitude of scattering/reflection at short distances is not very large. In contrast, when the ultrasonic probe 12 floats from the object surface, signals are generated (received) due to strong reflection at short distances and slowly attenuated. As shown in FIG. 8(b), therefore, while the ultrasonic transducer elements are partly floated from the object surface, large signals are received from the floating ultrasonic transducer elements (two elements in FIG. 8(b)) at short distances. The backend processing unit 27 excludes reception signals from the ultrasonic transducer elements floating from the object surface based on large signals at short distances which are generated partly in this manner. This can suppress unnecessary responses.

### APPLICATION EXAMPLE 4

The following is an application example of forming and comparing partial images upon a plurality of different delay/amplitude corrections and re-setting delay/amplitude conditions for transmission/reception in accordance with the comparison results.

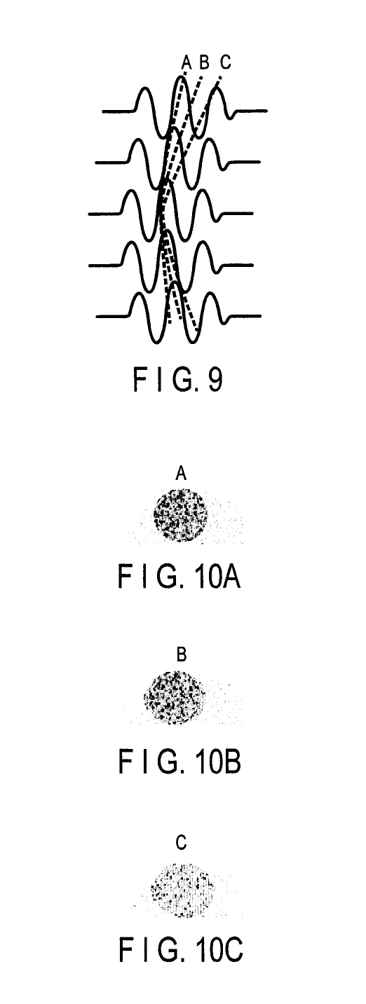

Assume that each ultrasonic transducer element actually receives a signal with a small time difference as compared with an arrival time difference B predicted from propagation times, as shown in FIG. 9. In this case, the apparatus forms partial images premised on three propagation delay times like those indicated by reference symbols A, B, and C in FIG. 9 by using the received signals for the respective ultrasonic transducer elements, and displays the resultant images as shown in FIGS. 10A, 10B, and 10C. The observer can re-set delay/amplitude conditions for transmission/reception by observing and comparing the displayed partial images and selecting an image corresponding to a desired delay time difference.

Comparing the examples shown in FIGS. 10A, 10B, and 10C (i.e., the cases of the arrival time differences A, B, and C shown in FIG. 9) leads to an image A with less blur at which the time differences match each other. This makes it possible to use conditions similar to actual time differences to change transmission/reception conditions for the formation of an image.

### APPLICATION EXAMPLE 5

Conditions similar to actual time differences may be changed to transmission/reception conditions for the formation of an image based on reception signals from a region of interest set on an ultrasonic image via the ultrasonic transducer elements.

### (Effects)

According to the above arrangement, the following effects can be obtained.

This ultrasonic diagnostic apparatus receives a plurality of reception signals for correction corresponding to the respective ultrasonic transducer elements via the same path as that for reception signals for the generation of an image, and analyzes the delay time differences and amplitude differences between elements, multiple reflection amounts, and the like based on the plurality of reception signals for correction. At least one of the transmission condition and the transmission/reception condition is corrected based on this result. It is therefore possible to optimize the transmission/reception condition with low consumption power and small circuit size by acquiring echo signals for the respective channels and separately receiving echo signals, and analyzing the inhomogeneity of the ultrasonic wave propagation medium. This makes it possible to provide an image with a high real-time property and little deterioration with respect to even a patient having a large inhomogeneous layer such as a fat layer.

### Second Embodiment

The second embodiment implements a transmission/reception condition optimization function by using a two-dimensional ultrasonic array probe.

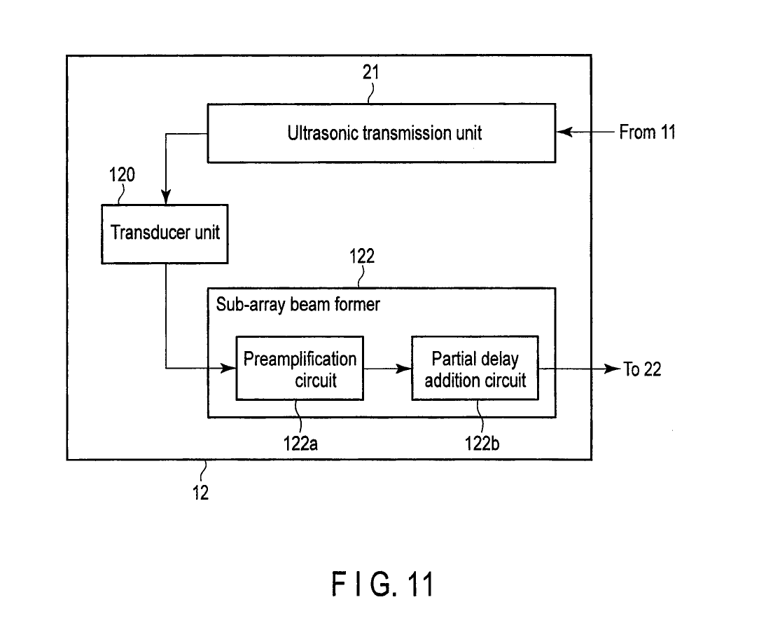

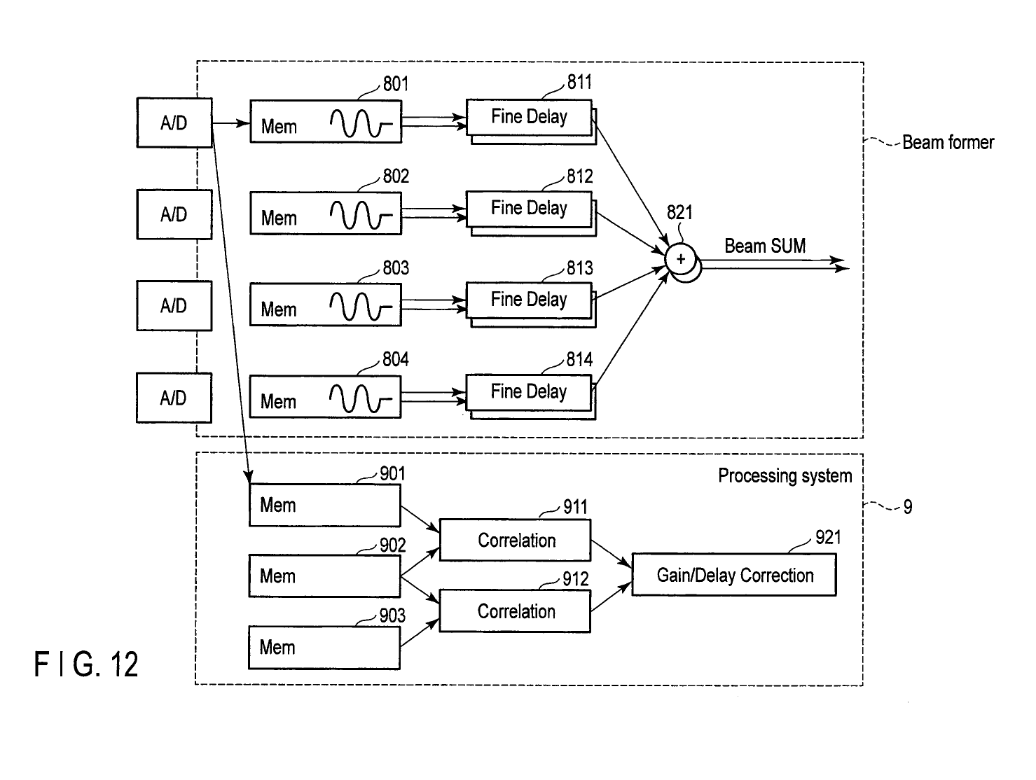

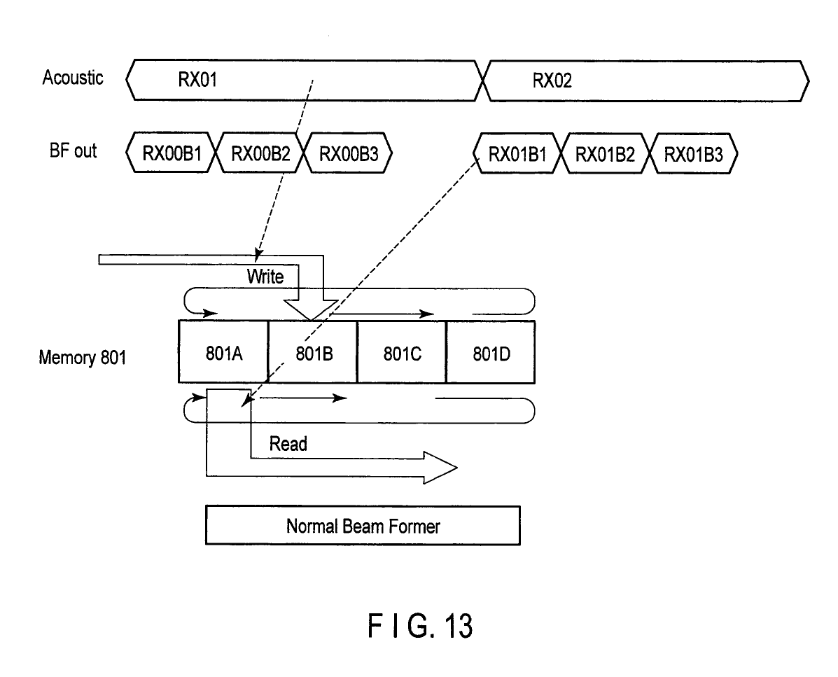

FIG. 11 is a block diagram of an ultrasonic probe 12 (two-dimensional ultrasonic array probe) according to this embodiment. As shown in FIG. 11, the ultrasonic probe 12 includes a transducer unit 120, a sub-array beam former 122, and an ultrasonic transmission unit 21.

An ultrasonic transmission unit 2 is the same as that shown in FIG. 1.

The transducer unit 120 includes a plurality of ultrasonic transducers arrayed in a two-dimensional matrix.

The sub-array beam former 122 includes a preamplification circuit 122a and a partial delay addition circuit 122b. The preamplification circuit 122a amplifies an echo signal for each channel which is received from the transducer unit 120. The partial delay addition circuit 122b partially delays and adds the amplified channel-based echo signals in units of several channels to ten-odd channels which are spatially adjacent to each other to generate a plurality of partial beams respectively corresponding to different local spaces in a scan area.

The echo signals as the plurality of partial beams generated by the partial delay addition circuit 122b are sent out to an ultrasonic reception unit 22 on the subsequent state which serves as a main beam former. The ultrasonic reception unit 22 executes transmission/reception condition optimization processing described in the first embodiment by using the plurality of echo signals received from the partial delay addition circuit 122b.

This embodiment has exemplified the arrangement having the ultrasonic transmission unit 21 and the sub-array beam former 122 provided on the ultrasonic probe 12 side. However, the embodiment is not limited to this, and may have an arrangement having both the ultrasonic transmission unit 21 and the sub-array beam former 122 provided on the apparatus main body 11 side or having either the ultrasonic transmission unit 21 or the sub-array beam former 122 provided on the apparatus main body 11 side. In addition, the embodiment has exemplified the delay amount control for focusing the data output from the beam former on a focus on straight lines having the same directivity. However, this focus need not be a focus on a continuous straight line, and it is possible to perform delay addition control by discontinuously designating only necessary places for imaging.

According to the arrangement described above, it is possible to implement a transmission/reception condition optimization function even by using a two-dimensional ultrasonic array probe.

Note that the present invention is not limited to the embodiment described above, and constituent elements can be modified and embodied in the execution stage within the spirit and scope of the invention. The following are concrete modifications.

Each function associated with each embodiment can also be implemented by installing programs for executing the corresponding processing in a computer such as a workstation and expanding them in a memory. In this case, the programs which can cause the computer to execute the corresponding techniques can be distributed by being stored in recording media such as magnetic disks ((floppy®) disks, hard disks, and the like), optical disks (CD-ROMs, DVDs, and the like), and semiconductor memories.

In addition, various inventions can be formed by proper combinations of a plurality of constituent elements disclosed in the above embodiments. For example, several constituent elements may be omitted from all the constituent elements disclosed in the above embodiments. Furthermore, constituent elements in the different embodiments may be properly combined.

### REFERENCE SIGNS LIST

1. An ultrasonic diagnostic apparatus comprising:
an ultrasonic probe including a plurality of ultrasonic transducer elements configured to transmit ultrasonic waves to an object in response to supplied driving signals and generate reception signals based on reflected waves from the object;
a transmission unit configured to supply the driving signals to the plurality of ultrasonic transducer elements in accordance with a predetermined transmission condition;
a reception unit configured to store a plurality of image generation reception signals corresponding to the plurality of ultrasonic transducer elements, generate a reception beam in accordance a predetermined reception condition by using each of the stored image generation reception signals, and store a plurality of correction reception signals corresponding to the plurality of ultrasonic transducer elements;
a correction unit configured to receive the plurality of correction reception signals and correct at least one of the transmission conditions and the reception condition based on the plurality of correction reception signals;
an image processing unit configured to generate an ultrasonic image based on the reception beam; and
a display unit configured to display the ultrasonic image.

2. The ultrasonic diagnostic apparatus of claim 1, wherein the reception unit comprises
a plurality of storage units provided for the respective ultrasonic transducer elements and including a first area used to store the image generation reception signal and a second area used to store the correction reception signal,
a plurality of first filters used for signal processing for the image generation reception signal corresponding to each of the ultrasonic transducer elements, and
a plurality of second filters used for signal processing for the correction reception signal corresponding to each of the ultrasonic transducer elements.

3. The ultrasonic diagnostic apparatus of claim 1, wherein the correction unit analyzes at least one of arrival time differences between the reception signals at the plurality of ultrasonic transducer elements, amplitude differences, and multiple reflection amounts at the plurality of ultrasonic transducers based on the correction reception signal for each of the ultrasonic transducer elements, and corrects at least one of the transmission condition and the reception condition based on the analysis result.

4. The ultrasonic diagnostic apparatus of claim 1, wherein at least one of the transmission condition and the reception condition is at least one of a sensitivity and delay time of each of the ultrasonic transducer elements.

5. The ultrasonic diagnostic apparatus of claim 1, wherein the correction unit analyzes amplitude differences between the reception signals at the plurality of ultrasonic transducer elements based on the correction reception signal for each of the ultrasonic transducer elements, and corrects a sensitivity of each of the ultrasonic transducer elements in accordance with the analysis result.

6. The ultrasonic diagnostic apparatus of claim 1, wherein the correction unit analyzes arrival time differences between the reception signals at the plurality of ultrasonic transducer elements based on the correction reception signal for each of the ultrasonic transducer elements, and corrects at least one of a transmission delay time and reception delay time of each of the ultrasonic transducer elements in accordance with the analysis result.

7. The ultrasonic diagnostic apparatus of claim 1, wherein the correction unit analyzes multiple reflection amounts at the plurality of ultrasonic transducer elements based on the correction reception signal for each of the ultrasonic transducer elements, and selects an ultrasonic transducer element to be used for the transmission and reception in accordance with the analysis result.

8. The ultrasonic diagnostic apparatus of claim 1, wherein the image generation unit generates a plurality of partial images in accordance with the plurality of transmission conditions and reception condition which are corrected by the correction unit,
the display unit displays the plurality of partial images, and
the correction unit corrects at least one of the transmission condition and the reception condition based on a partial image, of the plurality of displayed partial images, which is selected by the selection unit.

9. The ultrasonic diagnostic apparatus of claim 1, further comprising a setting unit configured to set a region of interest on an ultrasonic image displayed on the display unit,
wherein the correction unit corrects at least one of the transmission condition and the reception condition based on each reception signal from the region of interest at the ultrasonic transducer element.

10. The ultrasonic diagnostic apparatus of claim 1, wherein the correction unit receives the plurality of correction reception signals via the same path as that for the plurality of image generation reception signals.

11. The ultrasonic diagnostic apparatus of claim 1, further comprising a control unit configured to control the reception unit so as to switch between a first function of outputting the generated reception beam and a second function of outputting the corrected reception signal at a predetermined timing.

12. The ultrasonic diagnostic apparatus of claim 11, wherein the control unit controls the reception unit so as to time-divisionally store the plurality of image generation reception signals and the plurality of correction reception signals.

13. The ultrasonic diagnostic apparatus of claim 11, wherein the control unit controls the reception unit so as to store the plurality of image generation reception signals and the plurality of correction reception signals in physically different memory areas and output the signals at different timings.

14. The ultrasonic diagnostic apparatus of claim 1, wherein the ultrasonic probe comprises a two-dimensional ultrasonic probe.

15. An ultrasonic diagnostic apparatus control method comprising:
supplying driving signals, in accordance with a predetermined transmission condition, to a plurality of ultrasonic transducer elements configured to transmit ultrasonic waves to an object in response to supplied driving signals and generate reception signals based on reflected waves from the object;
storing a plurality of image generation reception signals corresponding to the plurality of ultrasonic transducer elements, generating a reception beam in accordance a predetermined reception condition by using each of the stored image generation reception signals, and storing a plurality of correction reception signals corresponding to the plurality of ultrasonic transducer elements;
receiving the plurality of correction reception signals and correcting at least one of the transmission condition and the reception condition based on the plurality of correction reception signals;
generating an ultrasonic image based on the reception beam; and
displaying the ultrasonic image.
